(19) United States
(12) Patent Application Publication
(10) Pub. No.: US 2024/0100360 A1
(43) Pub. Date: Mar. 28, 2024

(54) RADIATION TREATMENT PLAN OPTIMIZATION APPARATUS AND METHOD

(71) Applicant: Siemens Healthineers International AG, Steinhausen (CH)

(72) Inventor: Esa Kuusela, Espoo (FI)

(21) Appl. No.: 17/954,623

(22) Filed: Sep. 28, 2022

(51) Int. Cl. A61N 5/10

(52) U.S. Cl. CPC A61N 5/1031; A61N 5/1039

(57) ABSTRACT

A control circuit accesses computed tomography images for a given patient and also accesses at least one image that includes an image of an artificial portion of the given patient. The control circuit then ascribes a density value to the artificial portion of the given patient and optimizes a radiation treatment plan for that given patient as a function of the computed tomography images, the image of the artificial portion of the given patient, and the density value ascribed to the artificial portion of the given patient to provide a resultant optimized radiation treatment plan.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

A planning target volume (sometimes also referred to as a patient target volume) is a recognized approach to handling uncertainty in the radiation therapy treatment planning process. Using this approach, the clinical target volume (for example, a given tumor) is expanded in size to take into account expected or at least potential time-based variations in patient anatomy or positioning. This expansion typically involves applying a pre-defined and typically constant margin to the clinical target volume to define the planning target volume.

While a generally successful approach, the foregoing can be more problematic when the clinical target volume has a boundary close to an external surface of the patient. In such a case, the foregoing planning target volume can extend outside of the patient's body. One typical prior art practice is to remove any such exterior portion of the planning target volume from consideration in the optimization process. By another approach, heuristic rules are applied (either as part of the optimization process or as a post-processing step) such that the fields (i.e., corresponding to multileaf collimator apertures directly or optimal fluence masks) are extended in regions where dose is delivered outside the body as defined in the planning CT image albeit through a region of the aforementioned margin.

While again useful in many application settings, such approaches are not necessarily satisfactory in all instances.

### BRIEF DESCRIPTION OF THE DRAWINGS

The above needs are at least partially met through provision of the radiation treatment plan optimization apparatus and method described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a screen shot as configured in accordance with various embodiments of these teachings;

FIG. 4 comprises a screen shot as configured in accordance with various embodiments of these teachings;

FIG. 5 comprises a screen shot as configured in accordance with various embodiments of these teachings; and

FIG. 6 comprises a screen shot as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a control circuit accesses computed tomography images for a given patient and also accesses at least one image that includes an image of an artificial portion of the given patient. The control circuit then ascribes a density value to the artificial portion of the given patient and optimizes a radiation treatment plan for that given patient as a function of the computed tomography images, the image of the artificial portion of the given patient, and the density value ascribed to the artificial portion of the given patient to provide a resultant optimized radiation treatment plan.

In many application settings, that artificial portion of the given patient has a real-world density of air. Nevertheless, by one approach, these teachings provide for ascribing a density value to that artificial portion that is greater than that of air. For example, these teachings can provide for ascribing the density value of water to the artificial portion. By one approach, this density value is uniformly ascribed throughout the artificial portion of the given patient.

These teachings are highly flexible in practice and will accommodate, for example, application settings where the artificial portion of the given patient lies completely outside the image of the given patient. This may occur, for example, wherein all or part of the artificial portion of the given patient includes a part of a planning treatment volume (such as when the artificial portion is wholly a part of a planning treatment volume that comprises a predetermined margin that has been added for the given patient).

By one approach, these teachings will accommodate employing a cost function that evaluates a candidate radiation treatment plan solution based upon the computed tomography images for the given patient against a candidate radiation treatment plan solution based upon the image of the artificial portion of the given patient and the density value ascribed to that artificial portion. That cost function may, by one approach, include a term that penalizes nonhomogeneous dose distribution in a planning treatment volume that extends outside a patient's body (for example, when the dose distribution in a planning treatment volume that extends outside a patient's body is calculated as a function of the image of the artificial portion of the given patient and the density value ascribed to the artificial portion of the given patient).

So configured, these teachings can better accommodate uncertainty in the radiation treatment planning process and in particular can better and more efficaciously take into account daily variations in patient anatomy and/or positioning even in situations where the clinical target volume is disposed close to the patient's skin. The present teachings have the advantage of presenting a systematic approach to accommodate such situations that avoids, for example, the rigid application of heuristic rules.

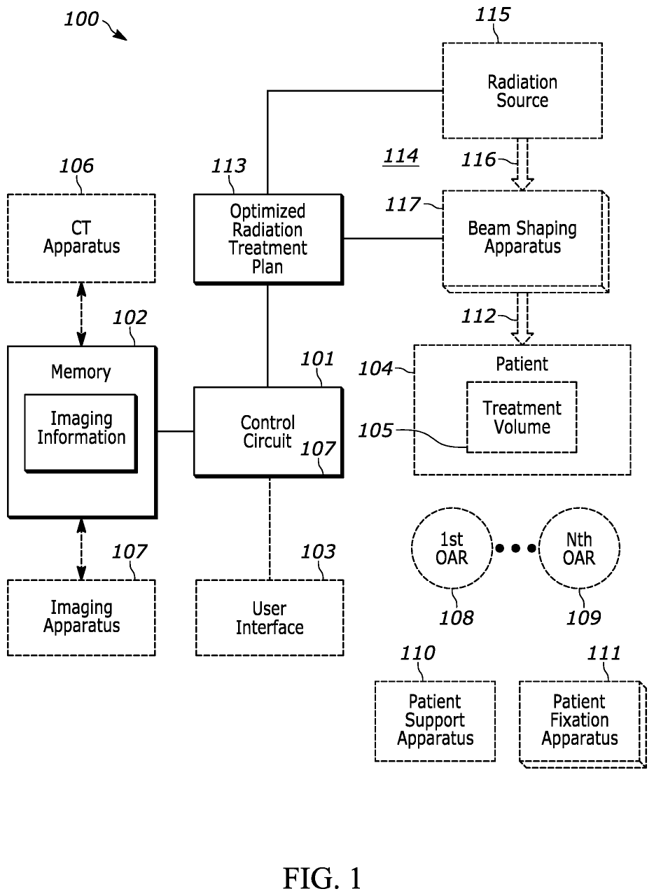

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as optimization information for a particular patient (such as, but not limited to, computed tomography images for that patient, and one or more images that include an image of an artificial portion of that patient) and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

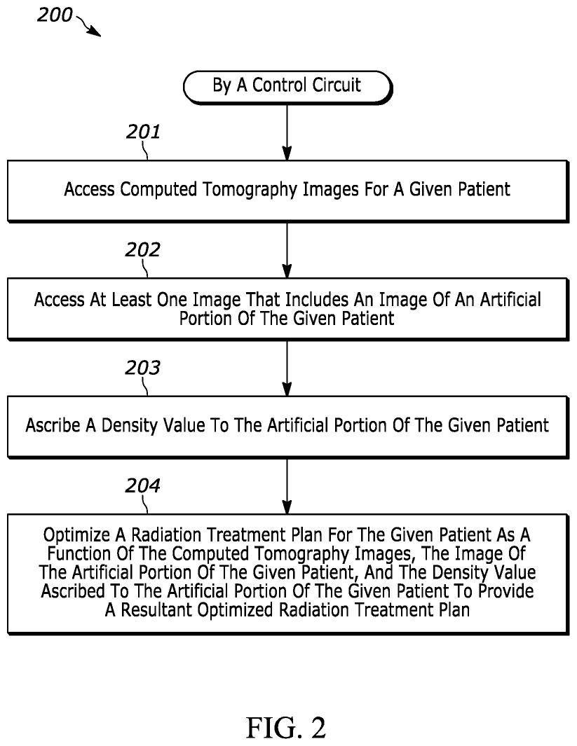

Referring now to FIG. 2, a process 200 that can be carried out, for example, in conjunction with the above-described application setting (and more particularly via the aforementioned control circuit 101) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

At block 201 of this process 200, the control circuit 101 accesses one or more computed tomography images for a given patient 104 (for example, by retrieving such images from the aforementioned memory 102). Computed tomography is a known procedure that uses a computer linked to an x-ray machine to make a series of detailed pictures of areas inside a patient's body. The pictures are taken from different angles and are used to create 3-dimensional views of tissues and organs.

At block 202 the control circuit 101 accesses at least one image that includes an image of an artificial portion of the given patient 104. By one approach that artificial portion partially or completely lies external to an image of the given patient 104 but is nevertheless treated as an artificial portion of that patient 104 for these purposes. In many useful application settings, at least a portion (and potentially all) of that artificial portion of the given patient 104 includes a part of a planning treatment volume. For example, that artificial portion may comprise a predetermined margin that has been added to a clinical target volume for the given patient to derive the planning treatment volume.

The image that includes an image of an artificial portion of the given patient 104 can be generated in any of a variety of ways. By one approach, the image comprises a computed tomography image that has been altered/edited/augmented to include the aforementioned artificial portion. That alteration/editing/augmentation may be effected by a human operator and/or automatically by the control circuit 101 as desired.

At block 203 the control circuit 101 ascribes a density value to the aforementioned artificial portion of the given patient. In one application setting that artificial portion has a real world density of air (where, for example, that artificial portion exists external to the patient's body). Notwithstanding that real world density of air, these teachings can provide for ascribing to that artificial portion a density that is greater than that of air. For example, by one approach, the ascribed density can be that of water.

These teachings are flexible in practice. By one approach, only part of the artificial portion is ascribed an alternative density. By another approach, all of the artificial portion is ascribed that alternative density. By one approach, different portions of the artificial portion are ascribed different alternative densities. By another approach, the alternative density is uniformly ascribed throughout the artificial portion of the given patient 104. (For example, although the artificial portion might have a real world density equal to the density of air, the density of water may be uniformly ascribed throughout that artificial portion.)

At block 204, the control circuit 101 then optimizes a radiation treatment plan for the given patient 104 as a function of the aforementioned computed tomography image(s), the aforementioned image of the artificial portion of the given patient, and the aforementioned density value ascribed to the artificial portion of the given patient to provide a resultant optimized radiation treatment plan 113.

By one approach, the foregoing optimization can comprise employing a cost function that evaluates a candidate radiation treatment plan solution based upon the computed tomography images for the given patient against a candidate radiation treatment plan solution that is based instead upon the image of the artificial portion of the given patient in combination with the density value that is ascribed to that artificial portion. Such a cost function may include a term that penalizes nonhomogeneous dose distribution in a planning treatment volume that extends outside a patient's body wherein the dose distribution in a planning treatment volume that extends outside a patient's body is calculated as a function of the image of the artificial portion of the given patient and the density value ascribed thereto. (As used in paragraph, the expression “extends outside” includes both a volume that is partially in the patient's body and partially external to the patient's body as well as to a volume that is wholly external to the patient's body.)

Additional details regarding these teachings will now be provided by way of some examples. It will be understood that the specific details of these examples are intended to serve an illustrative purpose and are not to be understood as suggesting any particular limitations with respect to these teachings.

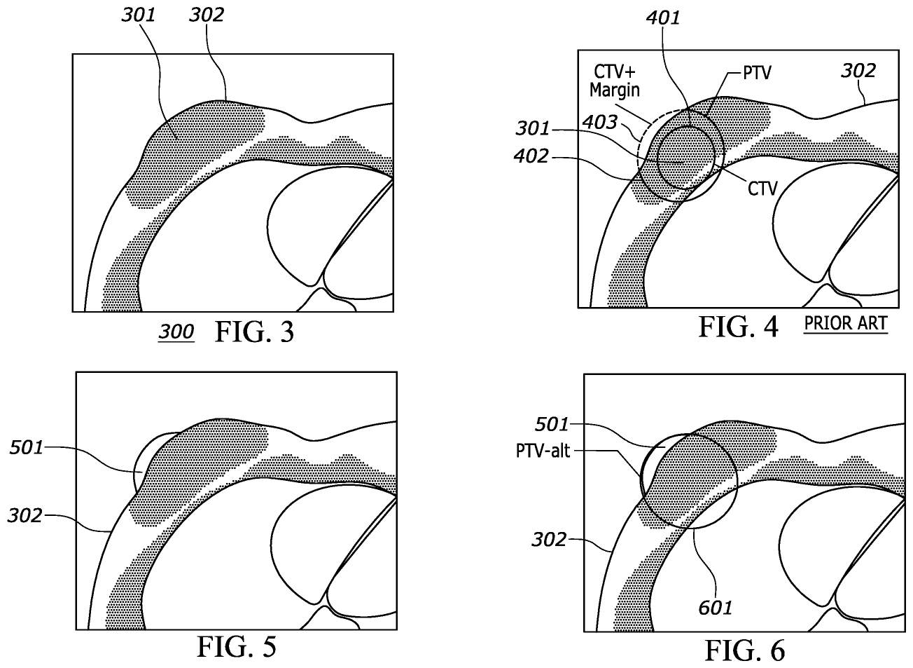

Generally speaking, these teachings address ways to account for situations where the planning treatment volume extends outside the patient's body. FIG. 3 presents an image that serves here as an illustrative example of a computed tomography image 300 for a given patient, and in particular, an original planning computed tomography image. In this case, the patient has a tumor 301 that is positioned very close to the edge/skin 302 of the patient. Referring to FIG. 4, an oncologist defined the clinical target volume as denoted by reference numeral 401.

In a typical prior art approach, a planning treatment volume is automatically created by adding a margin at a predetermined distance from the clinical target volume 401, with the exception that the planning treatment volume is not allowed to extend beyond the patient's body. Reference numeral 402 denotes the resultant planning treatment volume using this approach. Note that the dashed line denoted by reference numeral 403 represents where the planning treatment volume 402 would have extended but for that portion of the extended margin being located beyond the patient's skin 302. Per that prior art practice, the latter portion of the margin is excluded from what becomes the planning treatment volume 402. The optimization process then proceeds using that planning treatment volume 402.

Per the present teachings, and referring now to FIG. 5, this process uses an image that includes an image of an artificial portion of the patient. Continuing with the example begun above, this artificial portion is denoted by reference numeral 501 and constitutes that portion of the planning treatment volume described above that was cut away for lying outside the patient's body.

In addition, these teachings provide for assigning a non-air density to that artificial portion 501. In a typical application setting this assigned density may comprise a reasonable material density in the context of the patient's body. The density of water can serve well in many application settings. As another example, these teachings will accommodate copying a density value for a nearest point within the patient's body. As yet another example, these teachings will accommodate using a model-based approach that copies (to a greater or lesser extent) the density of some relevant point of comparison.

Per these teachings, are referring now to FIG. 6, the resultant planning treatment volume as denoted by reference numeral 601 includes both the volume encompassed by the above-described prior art approach as well as the artificial portion 501. The optimization process then proceeds using that resultant planning treatment volume 601.

As noted above, the optimization process can utilize a cost function that evaluates candidate solutions for both the prior art planning treatment volume (i.e., as shown in FIG. 4) and the resultant planning treatment volume (i.e., as shown in FIG. 6). Such an approach can be useful because the new image will not assuredly represent the most probable situation at the time of actually administering the radiation to the patient. By one approach, plan creation should still be based primarily on plan quality as measured with respect to the original planning computed tomography image. In practice, that can comprise using the original cost function defined by clinicians that contain all contributions from critical organs and using the cut-away planning treatment volume of FIG. 4 to evaluate target dose distribution. The cost function can then be augmented by adding an additional term that penalizes non-homogeneous dose distribution in that portion of the planning treatment volume that extends outside of the patient's body where the dose distribution can be calculated using the alternative image.

It may be beneficial for that additional penalty term to be relatively low so that it does not dominate the optimization. Generally speaking, it may be beneficial for the augmented cost function to drive the solution to open the corresponding multileaf collimator leaves and/or to add non-zero fluence into regions where the field includes air in the original planning computed tomography image but not altering parts of the solution that are contributing to the dose calculated using the original planning computed tomography image.

By one approach, it may be useful in some application settings to effect any target masking based only on the larger, non-cropped planning treatment volume structure.

By one approach, it may be useful in some application settings to apply these teachings in situations where part of the planning treatment volume extends to a region where the density is much lower than the density in the clinical target volume even if that region is otherwise covered by the patient's body. The foregoing situation may include, for example, cavities within the patient's body.

These teachings will also accommodate, if desired, generating more than one alternative image (having, for example, alternative corresponding planning treatment volumes) that represent different ways the patient's anatomy may be presented during treatment. In such a case, these teachings will accommodate assigning different weights to each planning treatment volume variant to reflect the relative probability of the corresponding presentation occurring.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A method comprising:
by a control circuit:
accessing computed tomography images for a given patient;
accessing at least one image that includes an image of an artificial portion of the given patient;
ascribing a density value to the artificial portion of the given patient;
optimizing a radiation treatment plan for the given patient as a function of the computed tomography images, the image of the artificial portion of the given patient, and the density value ascribed to the artificial portion of the given patient to provide a resultant optimized radiation treatment plan.

2. The method of claim 1 wherein the artificial portion of the given patient has a real world density of air.

3. The method of claim 2 wherein the density value that is ascribed to the artificial portion of the given patient is greater than that of air.

4. The method of claim 3 wherein the density value that is ascribed to the artificial portion of the given patient is that of water.

5. The method of claim 1 wherein the density value that is ascribed to the artificial portion of the given patient is uniformly ascribed throughout the artificial portion of the given patient.

6. The method of claim 1 wherein the artificial portion of the given patient all lies external to an image of the given patient.

7. The method of claim 1 wherein at least a portion of the artificial portion of the given patient includes a part of a planning treatment volume.

8. The method of claim 7 wherein the artificial portion of the given patient is wholly a part of a planning treatment volume that comprises a predetermined margin that has been added to a clinical target volume for the given patient.

9. The method of claim 1 wherein optimizing a radiation treatment plan for the given patient as a function of the computed tomography images, the image of the artificial portion of the given patient, and the density value ascribed to the artificial portion of the given patient to provide a resultant optimized radiation treatment plan comprises employing a cost function that evaluates a candidate radiation treatment plan solution based upon the computed tomography images for the given patient against a candidate radiation treatment plan solution based upon the image of the artificial portion of the given patient and the density value ascribed to the artificial portion of the given patient.

10. The method of claim 1 wherein the cost function includes a term that penalizes non-homogenous dose distribution in a planning treatment volume that extends outside a patient's body wherein the dose distribution in a planning treatment volume that extends outside a patient's body is calculated as a function of the image of the artificial portion of the given patient and the density value ascribed to the artificial portion of the given patient.

11. An apparatus comprising:
a control circuit configured to:
access computed tomography images for a given patient;
access at least one image that includes an image of an artificial portion of the given patient;
ascribe a density value to the artificial portion of the given patient;
optimize a radiation treatment plan for the given patient as a function of the computed tomography images, the image of the artificial portion of the given patient, and the density value ascribed to the artificial portion of the given patient to provide a resultant optimized radiation treatment plan.

12. The apparatus of claim 11 wherein the artificial portion of the given patient has a real world density of air.

13. The apparatus of claim 12 wherein the density value that is ascribed to the artificial portion of the given patient is greater than that of air.

14. The apparatus of claim 13 wherein the density value that is ascribed to the artificial portion of the given patient is that of water.

15. The apparatus of claim 11 wherein the density value that is ascribed to the artificial portion of the given patient is uniformly ascribed throughout the artificial portion of the given patient.

16. The apparatus of claim 11 wherein the artificial portion of the given patient all lies external to an image of the given patient.

17. The apparatus of claim 11 wherein at least a portion of the artificial portion of the given patient includes a part of a planning treatment volume.

18. The apparatus of claim 17 wherein the artificial portion of the given patient is wholly a part of a planning treatment volume that comprises a predetermined margin that has been added to a clinical target volume for the given patient.

19. The apparatus of claim 11 wherein the control circuit is configured to optimize a radiation treatment plan for the given patient as a function of the computed tomography images, the image of the artificial portion of the given patient, and the density value ascribed to the artificial portion of the given patient to provide a resultant optimized radiation treatment plan by employing a cost function that evaluates a candidate radiation treatment plan solution based upon the computed tomography images for the given patient against a candidate radiation treatment plan solution based upon the image of the artificial portion of the given patient and the density value ascribed to the artificial portion of the given patient.

20. The apparatus of claim 11 wherein the cost function includes a term that penalizes non-homogenous dose distribution in a planning treatment volume that extends outside a patient's body wherein the dose distribution in a planning treatment volume that extends outside a patient's body is calculated as a function of the image of the artificial portion of the given patient and the density value ascribed to the artificial portion of the given patient.
